(19) United States
(12) Patent Application Publication
(10) Pub. No.: US 2011/0301101 A1
(43) Pub. Date: Dec. 8, 2011

(54) T cell immune balance peptides

(76) Inventor: Shuqi Xia, Guangzhou (CN)

(21) Appl. No.: 12/892,902

(22) Filed: Sep. 28, 2010

(30) Foreign Application Priority Data
Jun. 7, 2010 (CN) 201010196132.8

(51) Int. Cl. A61K 38/16; A61K 38/05; A61K 38/08; A61K 38/06; A61P 37/00; A61K 38/10

(52) U.S. Cl. 514/21.3; 514/21.4; 514/21.5; 514/21.6; 514/21.7; 514/21.8; 514/21.9; 514/21.91

(57) ABSTRACT

This invention is related to a peptides mixture. Without the need to consider the patients' genetic background, it can interfere with MHC-pathogenic peptide-TCR formation, which includes the interference with pathogen peptide binding with MHC and pMHC binding with specific TCR, suppression of the immune synapse formation when the specific T cell immune response occurs, reduction of the number and density of the MHC-specific immune response mediated pathogenic peptide-TCR in the immune synapse, and suppression of the highly activated signal transduction in the immune synapse. Hence it can negatively regulate the T cell specific immune response, reduce the specific immune cell activation, proliferation and effect, and make the radical T cell specific immune response more stable and last longer. It can be used to treat diseases with excessive T cell-specific immune reaction, such as severe flu, SARS, hand-foot-and-mouth disease, viral pneumonia, bacterial infections, severe autoimmune disease and etc.

The present application claims priority to CN201010196132.8, China, which was filed on Jun. 7, 2010.

### FIELD OF THE INVENTION

This invention relates to a peptides mixture, which can negatively regulate T cell mediated specific immune response induced by protein and other macromolecular antigens, viruses, bacteria, especially when the T cell specific immune response is very strong. It enables T cell specific immune response to proceed in a more moderate and sustainable manner.

### BACKGROUND OF THE INVENTION

For the treatment of hypersensitivity, autoimmune disease, graft rejection, inflammation and other immune hyperfunction diseases, the drugs include: \textcircled{1} non-specific immunosuppressive agents: such as chemical agents (alkylating and antimetabolite), hormones, metabolic products of fungi (cyclosporin A and FK-506) and some Chinese medicinal materials. Most of these agents have obvious toxicity and side effects, mainly used in organ transplant rejection, which can lead to decreased immune function, increase the chance of infection, and long-term applications may induce tumors. \textcircled{2} the antibodies of lymphocytes and their surface molecules, which is used for the treatment of anti-allograft rejection, autoimmune diseases, such as the CD3 monoclonal antibody treat the acute heart, liver, kidney transplant rejection, the anti-CD4 monoclonal antibody treat RA and other autoimmune diseases.

Recently occurred viral infections, such as SARS (severe acute respiratory syndrome), is a new respiratory disease, caused by a kind of coronavirus, accompanying with the main symptoms such as fever, dry cough, chest tightness. It can rapidly progress into severe respiratory system failure, since it is highly contagious, and rapidly progressive. The disease has an incubation period of a week or two, and then progresses rapidly. It is very important for the patient to induce an effective specific T cell immune response for the clearance of the virus. While the specific immune response induced by SARS (including Th1-related cellular immunity and Th2-related humoral immunity) is excessively intense, leading to the extensive usage of a large dose of glucocorticoid. The widespread used of glucocorticoids has a wide range of inhibitory effect on the innate immunity and the adaptive immunity, which has negative effects on the control of virus infection. Mycoplasma, chlamydia, streptococcus pneumoniae and other microbial infections may secondary to the immunosuppressive treatment, increasing the lung injury. In addition to leading to death in many cases, the disease also caused many severe sequelas, such as severe lung function impairment, necrosis of femoral head. SARS can infect the lungs, kidneys and gastrointestinal epithelial cells, but there are no reports of chronic infection. Infected children are usually with a better prognosis. Young adults generally have more severe symptoms of infection which may be associated with high immune responsiveness.

It is of a strong practical significance to explore how to develop an immunosuppressive drug with better specificity, moderation and safety, to against relapsed virulent infections.

### BRIEF SUMMARY OF THE INVENTION

The present invention introduces the immune mutual selection theory to deal with the interactions among the three: MHC, peptides, TCR. T cell mediated specific immune response induced by pathogens or molecules, represented by the TCR with maximum affinity to their formed peptide-MHC or a small group of high-affinity TCR, can be interfered by the low affinity peptide-MHC, resulting in reducing the formation of the MHC-pathogen peptide-TCR (pathogen-related high-affinity TCR group). Meanwhile, the formed MHC-low affinity peptide-TCR (pathogen-related high-affinity TCR group) can inhibit the formation of the pathogen-specific T cell immune synapse, reduce the number of the formed synapse, cut down and interfere with the transmission of the activated signals.

The peptides which form the structure of the low affinity peptide-MHC may also be competitively inhibit the pathogenic peptides binding to MHC, reducing the formation of pathogenic peptide-MHC, cutting down the presenting speed of pathogen peptides. Therefore, it can inhibit the pathogen-specific T cell immunity, and extend the time of the specific T cell immune response.

The peptides can be synthesized in a random sequence, which can form low affinity peptide-MHC in the human body. Sources of those peptides are mostly extracted from the vertebrate proteins. Because the proteins from those animals are the result of the selection by their own TCR and MHC, the peptides formed by them mediate a lower average affinity compared with other pathogenic microorganisms in human body. That is: the average affinity of the MHC-peptide derived from a vertebrate-TCR (pathogen-related high-affinity TCR group)<the average affinity of the MHC-peptide derived from a vertebrate-TCR (vertebrate-related high-affinity TCR group)<the average affinity of the MHC-pathogen peptide-TCR (pathogen-related high-affinity TCR group). Therefore, peptides derived from those sources can be used to negatively regulate the specific T cell immune response induced by pathogenic microorganisms. Peptides extracted from human proteins can negatively regulate the specific T cell immune response caused by the body's own high affinity peptides which can induce autoimmune diseases.

The peptides extracted from the animals can be prepared by chemical or genetic engineering approach after structural analysis. The peptides are used to treat the diseases with excessive T cell-specific immune response, such as pathogenic microbial infection and severe autoimmune disease, etc. These peptides can be combined with related antibiotics or antiviral drugs during the treatment of pathogenic microorganism infection.

### BRIEF DESCRIPTION OF THE DRAWINGS

FIG. 1. The transduction of TCR-pMHC signal induce immune synapse formation through the T cell cytoskeleton rearrangement, taken from “Principles of immunity,” page 144, edited by Guangyan Zhou, published by Shanghai Science and Technology Publishing House.

FIG. 2. Immune synapse makes the formation of functional affinity within the multiple TCR-pMHC molecules, which causes the activation of T cells, taken from “Principles of immunity,” page 144, edited by Guangyan Zhou, published by Shanghai Science and Technology Publishing House.

FIG. 3. The changing process of the TCR structure: under the influence of progressively increasing of the pMHC, the TCR structure transforms from the same body to the similar body, and terminally forms the different body.

FIG. 4: On the horizontal axis (affinity q), five loci in the similar body (f1), C, E will lead to exclusion of the mutual selection, while A, B, D will not.

FIG. 5: The mutual selection between MHC and the peptides from a certain protein.

FIG. 6: The comparison chart about 14 peptides with different affinity to MHC and their corresponding ability in promoting Th proliferation, taken from “Principles of immunity,” page 8, edited by Guangyan Zhou, published by Shanghai Science and Technology Publishing House.

FIG. 7: The affinity between the TCR and other peptide groups from different sources, which has high-affinity to the core antigen group, is arranged in the coordinate of the mutual selection.

FIG. 8: Schematic diagram about the background distribution of the pMHC under the co-existence curve of a certain TCR in health human body.

FIG. 9: Schematic diagram about the mutual selection between the pMHC of the hepatitis B virus core antigen and its corresponding specific TCR when the immune activation occurs.

FIG. 10: Schematic diagram about the mutual selection between the pMHC of the hepatitis B virus core antigen and its corresponding specific TCR when the immune cytotoxicity occurs.

FIG. 11: Schematic diagram about the effect of the low-affinity peptide group J on the mutual selection between the high-affinity peptide group C and the specific TCR.

FIG. 12: Comparison of the promoting T cell proliferation activation among the antagonistic peptide 99R, the null peptide 99A and the activation peptide MCC.

FIG. 13: Antagonist peptides and null peptides can block T cell effector function at limiting agonist densities. 5C.C7 T cell blasts were incubated with CH27-ICAM-GFP B cells with the indicated peptide concentrations for 56 h, after which IL-2 levels were quantified by ELISA.

FIG. 14: A model for a narrow kinetic range for TCR antagonism. The curve represents a hypothetical distribution of pMHC complexes as a group of ligands for a given TCR. Moving from right to left starting with rare agonist peptides that optimally stimulate T cells, as T1/2 (half life of TCR-pMHC interaction) decreases, more peptides show weaker activation potential (increased f, frequency of peptide), eventually leading to a null phenotype where irrespective of their ability to participate in MHC clustering (Wulfing, et al, 2002), these peptides by themselves do not stimulate T cells. TCR antagonist peptides are postulated to occupy a narrow kinetic window between weak agonists and null ligands, with a sufficient time of interaction for an aberrant or incomplete signal, whereas null peptides do not bind long enough to elicit any signal.

FIG. 15: The effect of T cell immune balance peptide on the specific CTL. This figure was modified on the basis of the picture which is illustrated on page 3 in the third edition of “Medical Immunology”, edited by Weifeng Chen, published by People's Medical Publishing House.

### DETAILED DESCRIPTION OF THE INVENTION

When the heterologous proteins, viruses and bacteria are invaded the body, the self-protection immune response often can remove these extraneous material. But sometimes, the body will over-react, resulting in increased disease symptoms, even disability or death. Some special self-antigen can also induce the body to make excessive immune response, leading to the occurrence of autoimmune disease.

When these situations occur, the best is to make negative regulation in some extent. In order to overcome the strong and widespread immunosuppressive of the current agents, according to the development of immunology in the past ten years, the present invention provides a negative regulator to the specific immune response induced by T cell.

1, T Cell Specific Immune Response

T cells can not recognize the complete natural antigen molecule. The natural antigen need to be degraded into peptides by the antigen presenting cell (APC), and then bind to the major histocompatibility complex (MHC), and next moved to the cell surface of APC, and therefore it can be recognized by T cell. Antigens recognized by T cells are mainly presented by class I or II MHC molecules. The ternary TCR-pMHC complex is composed by the MHC, antigen peptide and TCR, which pMHC is the simplified form of “peptide-MHC”. TCR-pMHC complex is the most important molecular structure group, which can reflect the antigen specificity of the T cell recognition in T-APC interaction. Not only the antigenic peptides presented by Class I molecules and class II molecules are with different structures, but also the binding secondary receptor of proximal membrane domain in the two molecules are also different, which are CD8 and CD4 molecules, respectively, thus induces the following different patterns of antigen presentation: Class I molecules present endogenous antigens to CD8⁺ CTL for recognition; Class II molecules present exogenous antigens to CD4Th cell for recognition. The binding groove of class I molecule closes at both ends which accept 8 to 10 peptides. The N and C terminals of the peptides are buried in both ends of the slot. The antigen binding groove of II molecules is open at both ends, which can hold 13 to 25 long-chain peptides.

Three components in TCR-pMHC show a high degree of variability.

First of all, there are a great number of antigens. The antigen peptides enter the MHC antigen binding groove are not only from different antigen molecules, but also from different peptides in the same antigen. Its diversity is greatly incalculable.

Secondly, MHC variability results from two aspects: multiple genes and polymorphisms. For example, the classical antigen presenting molecules HLA, has two different classes, I and II molecules. The Class I molecules can also be divided into different loci products of HLA-A, HLA-B, HLA-C. Each of them constructs antigen-presenting molecule with different structures. Moreover, HLA is extremely rich in polymorphism. The classic class I and II molecules have a large number of alleles.

Thirdly, the diversity of TCR molecules is measured from 100 thousands to millions.

The TCR Receptor and the ligand pMHC are closely connected in the process of T cells recognition of antigen. There is also interaction between the antigen peptide and MHC molecules, the two components which constitute the ligand pMHC. Peptides generally contain two or more than two sites as anchor points binding to specific MHC molecule. Amino acids located on the site are known as anchor residues. The anchor residues insert into the small bag of the antigen binding groove of the MHC molecule, and bind with MHC molecule through hydrogen bond. The middle part of the peptide generally has some degree of eminence which can be used as T cell epitope for TCR recognition. The combination of an antigen peptides and particular MHC II molecules is the result of the attraction and repulsion between the amino acid residues of the peptides and the peptide binding groove.

The realization of the interaction between the antigen peptides and MHC molecules is determined by the MHC alleles molecular structural characteristics of antigen binding groove, whether the peptides getting into the binding groove are consistent with the common peptide motif that it accepts, and the location and type of the inhibitory residue that the peptides may show. The difference of MHC alleles (polymorphism) is reflected among individuals. Therefore, the different patterns of antigenic peptide-MHC interaction may be directly involved in the differences of different individuals' response to the same antigen, and even decide the genetic susceptibility to the disease caused by the same pathogen.

After the recognition of pMHC on the surface of APC, TCR complex quickly mobilizes a number of receptors and their intracellular signal transduction molecules to the T-APC contact area, thus forming the immune synapse.

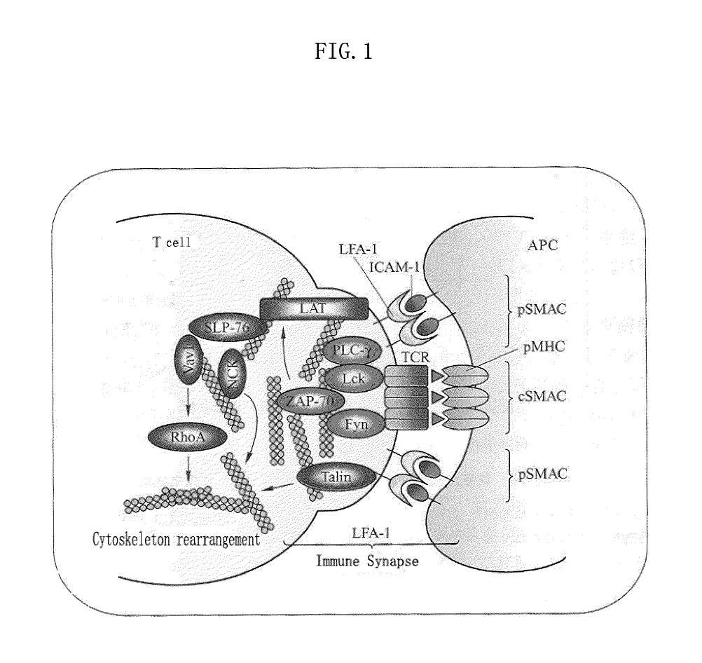

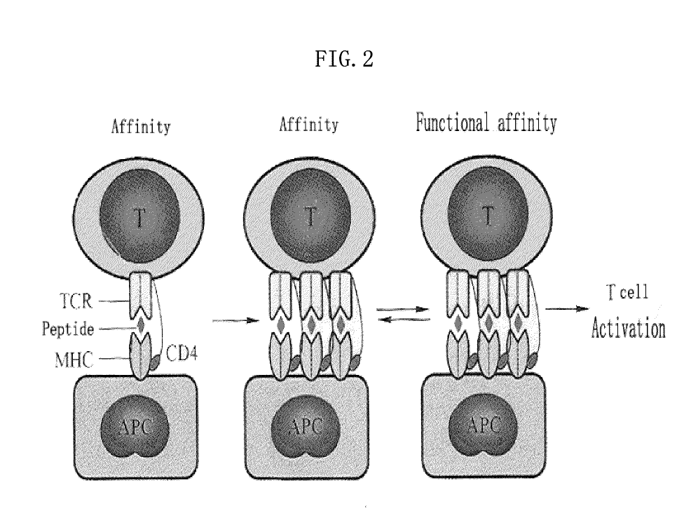

The significance of immune synapse formation: first, the aggregation of a number of TCR-pMHC trimer gives the opportunity for the affinity between a single receptor (TCR) and a single ligand (pMHC) evolving into a structural affinity. Structural affinity provides motivation strong enough for the corresponding signal transduction, and promotes T cell to perform its biological functions after antigen recognition, including proliferation, cytokine secretion and killing the target cells. It Involves in a variety of mechanisms: 1. A stable supramolecular structure makes multiple TCR molecules cluster transmit the T cell activation signal in parallel, promoting T cells fully activated. 2. The microstructure domain in the immune synapse narrows the T-APC interaction space, so that there is continuous TCR triggering opportunities. 3. TCR-pMHC complexes move to the center of the immune synapse so as to be condensed by 100 times, and T cells will not move in the balance force. 4. Immunological synapse provides the polarized interface for biochemical reaction and the platform for interaction between the molecules, which enhances the capacity for T cells to recognize antigen and also enhances the interaction between B7-1/B7-2 on APC and CD28 on T cell to generate costimulation signals, see FIG. 1 and FIG. 2.

TCR ligand pMHCs formed in nature partly act as an activation of the immune response and partly seem to play an antagonistic role. An article published on The Journal of Cell Biology in 2004, written by Cenk Sumen, makes a depth of research on the antagonistic effect of pMHC binding to TCR. The title is “T cell receptor antagonism interferes with MHC clustering and integrin patterning during immunological synapse formation”. In the foreword they wrote: T cell activation by nonself peptide—major histocompatibility complex (MHC) antigenic complexes can be blocked by particular sequence variants in a process termed T cell receptor antagonism. The inhibition mechanism is not understood, although such variants are encountered in viral infections and may aid immune evasion. Here, we study the effect of antagonist peptides on immunological synapse formation by T cells. We find that synapses formed on membranes presenting antagonist—agonist complexes display reduced MHC density, which leads to reduced T cell proliferation that is not overcome by the costimulatory ligands CD48 and B7-1. Most T cells fail to arrest and crawl slowly with a dense ICAM-1 crescent at the leading edge. Similar aberrant patterns of LFA-1/ICAM-1 engagement in live T-B couples correlate with reduced calcium flux and IL-2 secretion. Hence, antagonist peptides selectively disable MHC clustering and the stop signal, whereas LFA-1 valency up-regulation occurs normally.

From the above, the pMHC-TCR-mediated T cell-specific immune response is extremely complex.

2. The Mutual Selection Theory Explains the Interaction Between the MHC and Peptides as Well as pMHC and the TCR in T Cell-Specific Immune Response.

In order to facilitate the understanding of the interaction between MHC and peptide as well as the interaction between pMHC and the TCR and the consequence of T cell specific immune response, the inventor introduces the mutual selection theory of immunity.

For more in-depth understanding of the immune system, the inventor proposed the mutual selection theory of immunity: Organism selectively rejects the objects entering into its body, such as drugs, antigens, microorganisms, pathogenic cells or other allogeneic organisms cells, and such function is named anterograde selection; the objects entering into an organism, such as drugs, antigens, microorganisms, pathogenic cells or other allogeneic organisms cells, selectively rejects its body, and such function is named retrograde selection. The rejection of the party with increasing number in the mutual selection among all kinds of body cells and various components is named anterograde selection; the rejection of the other party is named retrograde selection.

Selective repulsion between the quantity and structure of each other will enable the other party (the structure determines function, characters, etc.) to change so that the two party tend to be composed of a stable system. The selective exclusion role of each other is decided by the respective quantity and structures, and this mutual role will lead to the survival of the fittest, those who not, to be modified or to be eliminated, and the end tends to implement immune mutual selection balance. As their number and structure of the system can be changed by the factors other than the system composed of the two parties, the stability of the both need to be achieved is in development and variation. The mutual selection is determined by the quantity and structures, and the fit ones exist, those unfit ones are to be modified or to be eliminated. Namely: yin yang is determined by the quantity and structures, balance, or xiang sheng xiang ke.

Objects with similar structure are called similar body. The composition of a Structure contained the type and the number of the similar bodies, and their relative position and relative motion. The relative motion between the similar bodies within the structure can alter the relative position, and thus leads the structure and also the internal structure of the similar bodies change. When the alteration is over a certain limit, the new similar body will be generated, and the number of the similar bodies will be changed.

The similarity of the similar body is descriptive in 0 to 1. When their difference can not be detected with all the current technology and methods, the similarity is equal to 1, named the same body. When the similarity of the same body close to 0, more than a certain value, you can think it is no longer the similar body. It can be classified into the different body.

The equation of the immune mutual selection:

F1+F2→S1+S2 (similarity of F1 close to 1, similarity of F2 close to 1, similarity of S1 close to 1 and similarity of S2 close to 1)

The reaction is generally irreversible. The two similar bodies on the left of the reaction are F1 and F2, as the xiang ke; S1 and S2 on the right of the reaction is xiang sheng after the xiang ke of F1 and F2. In the ordinary chemical equation, the left is also xiang ke, and the right is the xiang sheng product of the left xiang ke.

In a mutual selection system two selection sides are in a balanced co-existence. The reaction of the mutual selection in the cells or the macromolecules is usually an irreversible process. The reaction starts or stops, only in relation with the left of the F1 and F2. Therefore, the concentration (or frequency) and the mutual selection affinity of the two sides should have the following relationship:

F₁^{a}×F₂^{b}×Q=T (F₁ and F₂ is the concentration (or frequency); a, b is the combined potency; Q is the select affinity between the F1 and F2; T is the equilibrium constant of the mutual selection, also called co-existence constant, whose value may be affected by temperature)

The usual idea in immunology: D cells capture antigens in peripheral, gradually mature, and get into the lymph nodes, and then they can induce the activation of naive T cells. It seems to be natural in the life course. The mutual selection theory believe that: when DC cells mature, they will selectively exclude the naive T cells, especially the small part of the naive T cells that have a high selection affinity between them. They are the two objects with different structure that can not co-exist in the same system, which likes the relation of “irreconcilable, it was either you or me”. It will inevitably lead to structural transformation of each other, so that their frequency is decreased and simultaneously create new similar bodies.

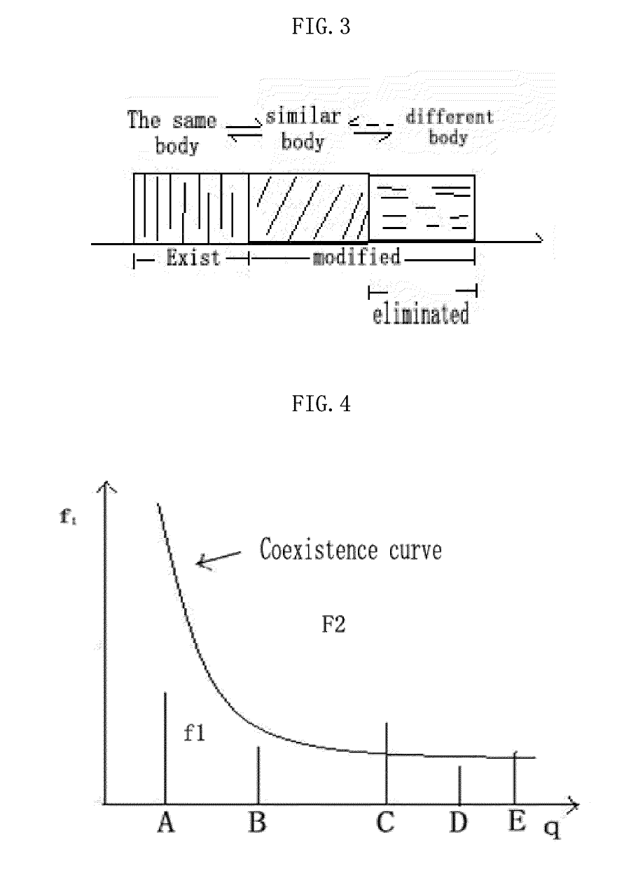

TCR is a macromolecule protein composed of two subunits. Each T cell has only one class of TCR. Under the selective exclusion role of the pMHC, the TCR with a multiple repetitive rate, some of which will alter in the space structure, and then the same body will transform into the similar body. When the pMHC frequency further increases, the same body will go on changing into the similar body, and then forms the structure of pMHC-TCR. pMHC induces the structural change of the same body TCR, so it happens the modified role. As the structure changes and the pMHC-TCR structure forms, so it happens the eliminate role and the same body TCR reduces its frequency. The changes are shown in FIG. 3.

In the mutual selection between pMHC and the TCR, F1 represents the same body pMHC in a particular individual, and F2 represents the same body TCR in a particular individual. Their co-existence in the mutual selection system will be restricted by the equilibrium constant, but the body belongs to non-uniform selection system. F₁ and F₂ represent the frequency rather than the concentration.

To simplify the equation F₁^{a}×F₂^{b}×Q=T, make a=b=1, and T=1, then there is F₁×F₂×Q=1. Transform the equation, and then there generate F₁=(1/F₂)×(1/Q). If the frequency of F2 in a individual is constant within a certain time, while the selection affinity between the F1 and F2 is determined by their owner structure, that is a fixed value, then F₁ is equal to (or less than) a certain value.

When the same body F1 expands to the other multiple same bodies pMHC (f1) similar to F1 in the individual, the affinity between the F2 and f1 is changed, then affinity is expressed as q. Meanwhile, the equation becomes:

f₁=(1/F₂)×(1/q), becomes a function of f₁ and q. It is like the function y=1/x. It is a hyperbolic curve in the coordinates. A certain specific pMHC in f1 has the only one affinity to the certain frequency F2. It means that the affinity between the different peptides with the same MHC and TCR is different. There is no overlap. If the affinity is same, then it must be mediated by the same peptide.

If the same body F2 is expanded to the other kinds of TCR (f2) similar to the F2, then f₁×f₂×q=1, and it is similar to the function xyz=1, like the shape of the china's ancient bell in the coordinate. It is complex and no longer be discussed here.

f₁=(1/F₂)×(1/q), The curve formed in the coordinate, called the balance curve of the mutual selection about F2, also known as the coexistence curve of F2. This means that the frequency of the F2 is F₂, at a certain affinity sites, the frequency of f1 is less than or equal to f₁. If it is more than that, the mutual selection occurs, triggering Xiang Ke eliminate effect, and generating the new similar bodies, as shown in FIG. 4.

The same individual has a relatively constant of MHC structure, which is different from the diversity of TCR. The interaction between MHC (M) and peptides group (p) whose length can be accepted by MHC is also in accordance with the mutual selection, that is:

p(the frequency of a single peptide)×M(MHC frequency)×q=T

In an individual, the structure of M is constant. When q is great, the structural features of this group with relatively fixed anchor point, which is only the extreme characteristics between the MHC and peptides in the mutual selection when the affinity is the highest, not representing the whole characteristics about the interaction between the p and M. As long as there is affinity, then when the M frequency is fixed, the frequency of some peptide increases, according to the balance equation of the mutual selection, and the structure of pMHC will come into being. Therefore, there is no need to consider the restriction of the anchor point of amino acids.

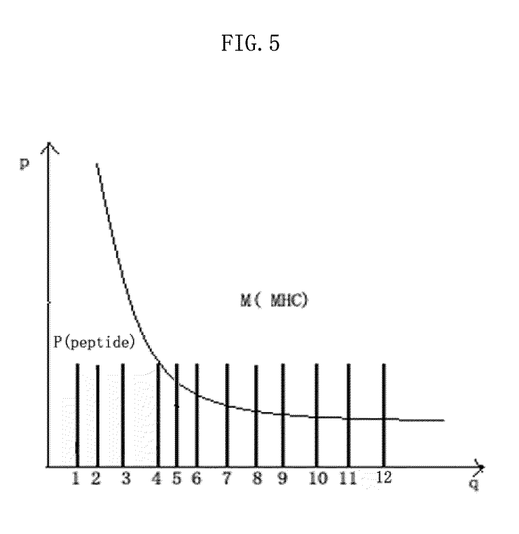

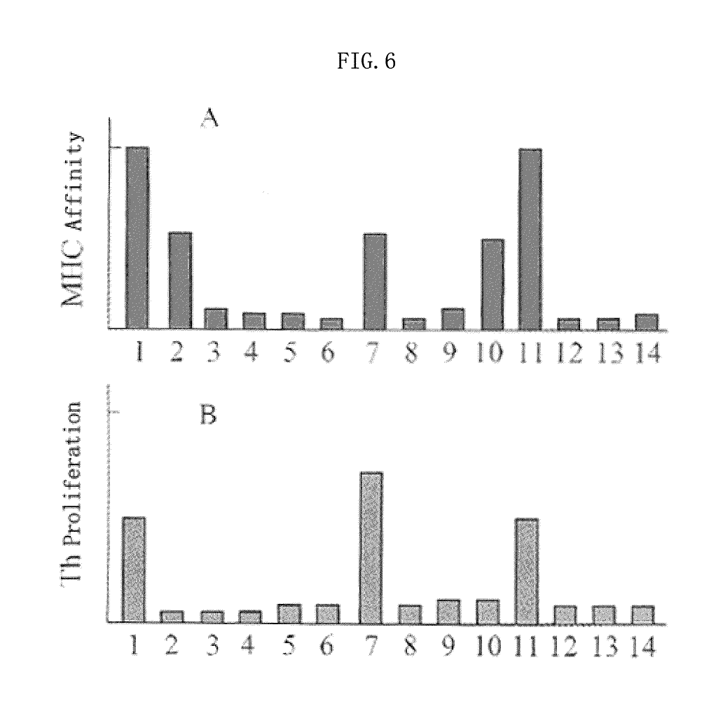

In FIG. 5, on the q-axis of affinity, the numbers from 1 to 12 are on behalf of the peptide fragments from some protein that can form the pMHC structures. If the number of the protein molecules is certain, the occurrence frequency of the peptides is the same. Their height is the same, but they are in turns increased the affinity to MHC. Suppose that the intracellular MHC frequency maintains constant by complementing timely expression, so that the coexistence curve does not shift. Thereby, the peptides with the No. 5, 6, 7, 8, 9, 10, 11, 12 can form the structures of pMHC, while the No. 1-4 can not. In order to make the No. 1-4 peptides form the structures of pMHC, it requires further increase the number of protein molecules, to increase their frequency, and to hit the curve, before the formation of pMHC structures. Within the peptides shown in the figure that can form the structures of pMHC, the peptide 12 generates the biggest quantity. Then the peptide 12 is the most representative for the affinity between the pMHC formed by the protein and some TCR. According to the level of affinity, the other 5-11 peptides are in the bilateral distribution around the peptide 12 as the center. The frequency is similar to the normal distribution and it is a kind of repetition frequency of the same body. In FIG. 6, the affinity between the peptide and MHC, and their biological activities are compared, reflecting the high MHC binding capacity is not necessarily accompanying with the highest biological activity, while the moderate affinity of three peptides have the higher or lower intensity of proliferation.

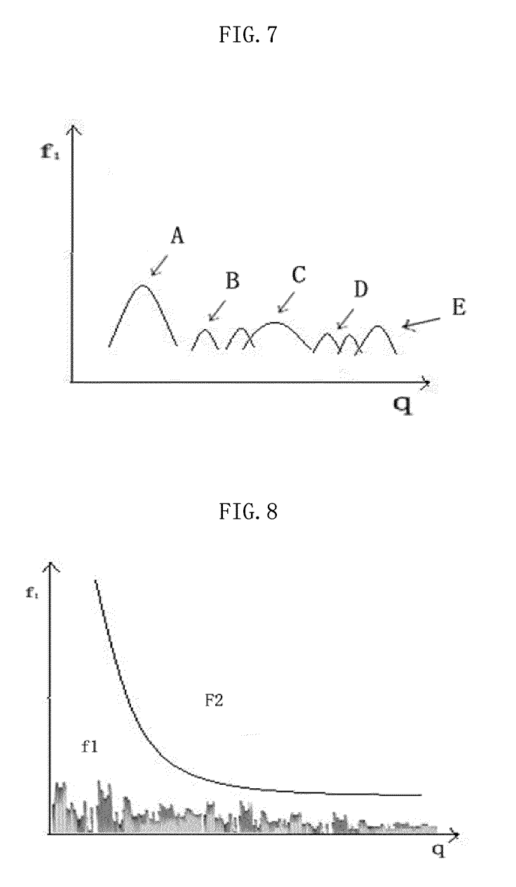

Meanwhile, in biology, some performance indexes of the same group, such as the height, weight, vital capacity in a group of children with the same age, and the output of crop growth under certain conditions, are generally subjected to normal distribution. The f1 in the bottom left and under the coexistence curve of F2, or the f1 in independent system without the constraint of the coexistence curve, the affinity frequency of the pMHC (f1) formed by the peptide of some viral protein and MHC to F2 presents in a normal distribution; the affinity frequency of the pMHC (f1) formed by a random mixture of very large numbers of the peptides and MHC to F2 presents in a normal distribution; the affinity frequency of the pMHC (f1) formed by the peptide of an individual's own protein and MHC to F2 presents in a normal distribution. The frequency of the affinity is a density frequency of the similar body. If a peptide-MHC group with repetition frequency and density frequency in normal distribution has the highest affinity with TCR, locating on the right most of q-axis, it must be able to mediate severe specific immune response induced by the high affinity. In some extent, it may be useful. However, if the duration of this response is too long or too strong, it will lead to illness or increase the disease. The peptide that mediates that reaction is called pathogen peptide. If F2 is represented the major TCR to mediate the specific immune response to hepatitis B virus core antigen in the individual, then the pMHC in normal distribution formed by the hepatitis B virus core antigen (HBcAg) should have the greatest affinity, that is to say its mean affinity locates in the most right side. The mean affinity of the other viral proteins is located on the left, so are randomly mixed peptides, and their bodies proteins are located in the far left, shown in FIG. 7.

In FIG. 7, E is on behalf of the pMHC formed by HBcAg, A represents the pMHC formed by the body's own proteins, B is on behalf of the pMHC formed by the animal protein closing to the human beings, C represents the pMHC formed by the mixture peptides with random amino acid sequence, D is on behalf of the pMHC formed by the proteins from other viruses and bacterial.

In the human body, in the F2 and f1 coexistence system, the f1 formed by the body's antigen, a variety of parasitic micro-organisms, slight infections, will be distributed under the mutual selection curve. Some affinity sites may appear in a high frequency and beyond the curve, causing a slight immune response, shown in FIG. 8.

To simplify the analysis, in the next analysis, the background distribution of f1 will no longer be considered. The following analysis of the mutual selection about the immune activation phase and cytotoxicity phase in the specific immune response, F2 is represented by the high affinity TCR mediated immune response to HBcAg for the analysis:

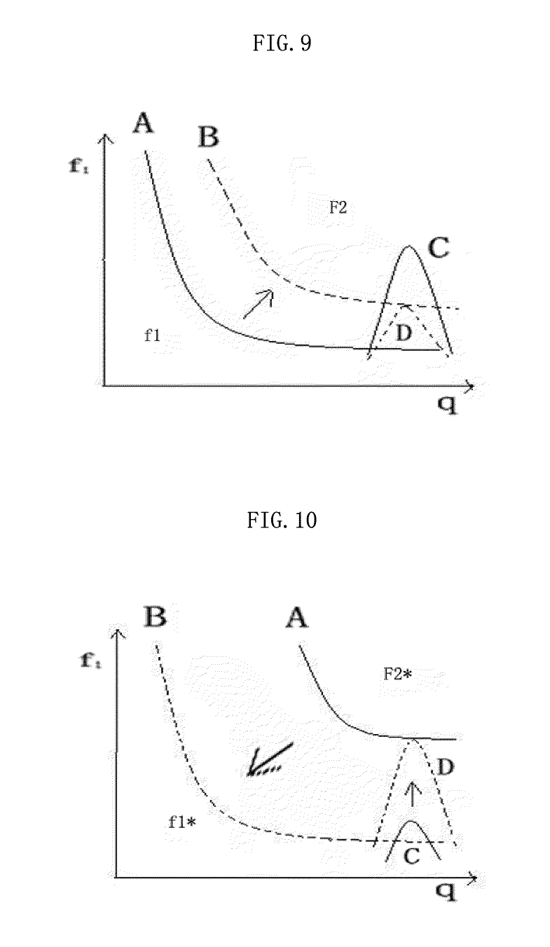

The formation of pMHC-TCR is in immune activation phase, as shown in FIG. 9.

As the hepatitis B virus infection, the viruses express more and more HBcAg in the human body, and the frequency curve of f1 (APC on the expression) in the normal distribution moves upward. First, do not consider the restriction of the coexistence curve. When the frequency curve (C) of f1 is higher than the coexistence curve, assuming both fixed the frequency and distribution in the mutual selection at this time, let to analyze the result of the mutual selection of two sides.

The frequency of F2 reduces in the mutual selection. The TCRs on the naive-type T cells are transformed into the TCRs on the activation T cells (Cenk Sumen, Michael L. Dustin, et al. T cell receptor antagonism interferes with MHC clustering and integrin patterning during immunological synapse formation. The Journal of Cell Biology, Volume 166, Number 4, Aug. 16, 2004 579-590). In the article, the majority of binding T cells after isolated can not be combined again, indicating the structure has been changed. Although their primary structure has not been changed, but the spatial structure has certainly been changed as the different body. The coexistence curve moves to the upper and right at this time, and the original normal distribution curve (C) moves down. In fact, in the short period of the immune reaction, the frequency of F2 is certain, but the normal distribution curve of HBcAg in the early stage of immune reaction has always been upward until the virus replication and expression are inhibited by a large extent, while the coexistence curve (A) of F2 moves upper and right. This dynamic change is called the chase-type mutual selection mode. A large number of monoclonal T cells, including Th1, Th2 and CTL, and also a large number of cytokines, such as IFN-gamma, TNF, etc., are induced in the activation to promote B cells to produce a large number of antibodies. The TCR of the activated T cells, although with the same primary structure to the initial T cells, but their spatial structure, must not be the same before and after activation, certainly with the different affinity to the same pMHC on the APC. The TCR on the activated T cells forms F2*in the effect stage. With a large number of cytokines, it is conducive for the virus-infected cells to form the HBcAg pMHC as f1*. * indicates they and the similar bodies in activation phase were belonged to different bodies.

The formation of pMHC-TCR is in immune effect phase. The MHC in the middle is mediated by class I molecules, as shown in FIG. 10.

In the process of the Hepatitis B virus infection, the viruses in the liver cells are ongoing replication and express a variety of protein, and also they stimulate the body's immune system to release large amounts of cytokines, especially IFN-gamma, which increases the expression of MHC. Not to consider the restriction of the coexistence curve, the frequency curve (C) of f1* (expression on the liver cells) in normal distribution moves up, and the frequency of F2* significant increases, so the coexistence curve of (A) moves quickly to the bottom and left. This dynamic change of the both is called the mutual assault-type mutual selection mode. When the cells are infected by the viruses, the specific immune responses are generally starting after 96 hours. At this time, the intracellular viruses have been a significant replication and expression, and the distribution curve of the f1* has been moved up a lot. Meantime, the frequency of F2* at this time is greatly increased, and the coexistence curve is quickly moved down, leading to the severe immune responses. A large number of infected cells are killed by granzyme, perforin, Fas, TNF or other effects. While a large number of inflammatory substances are released, they cause congestion and swelling in the infection site. Subsequently the tissue ischemia, hypoxia and ischemia—reperfusion injury lead to severe tissue necrosis and lower local immunity, so many secondary infections may occur, which can induce severe cases, even deaths.

Under the action of the innate immunity and the cytokines such as IFN-γ, the patients with acute hepatitis B can clear the hepatitis B viruses in the liver without cell damage and apoptosis. But then a large number of specific T cells and non-specific T cells come and lead to a lot of inflammation and the damage of the liver cells, even the severe situation. Although the viruses or their DNA have been undetected, but the accumulation of viral proteins and the relevant hydrolyzed peptides are remain in the infected cells. At this point, a lot of them are still be presenting by the MHC. The peak value of the CTL killing effect is mediated by the specific cross-linker of pMHC-TCR. This effect needs not happen again in a large extent, or do not need happen in a high-intensity.

3, The Immune Mutual Selection Theory Used in the T Cell Specific Immune Response: The Production of T Cell Immune Balance Peptides.

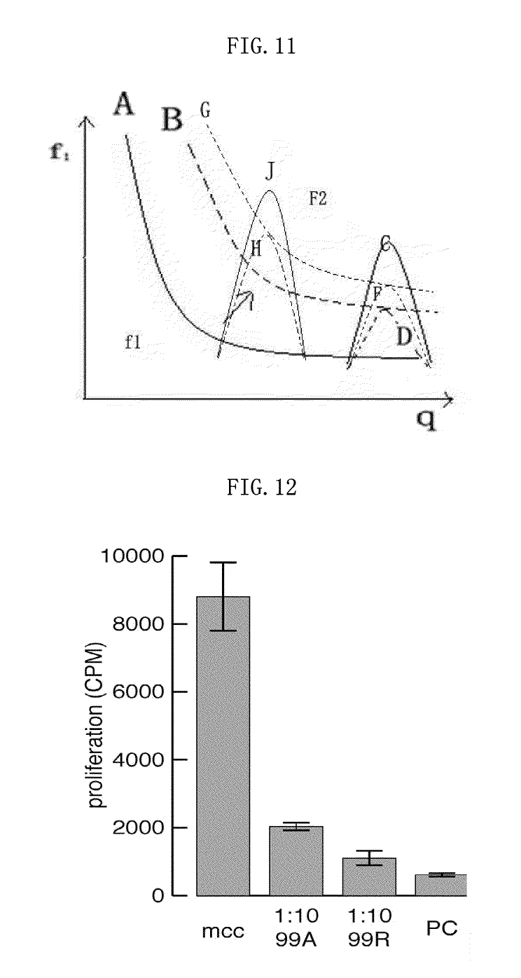

As shown in FIG. 7 and FIG. 9, the TCR mediated HBcAg specific T cell immune response, with a relative high affinity to the pMHC formed by HBcAg, forms the immune synapse with highly efficient activation, and activates T cell specific cellular immune response. If a group of pMHC with low affinity in the left of E group shown in FIG. 7, named J, appears in FIG. 9, thus forms the situation shown in FIG. 11.

With the emerged J group, the coexistence curve will move from A to G, then the distribution curve C will decline to the position F, not the original location of D. So, the frequency of the C group involves in the response will be greatly reduced, reducing the formation of the HBcAg-related high-affinity pMHC-TCR, and thus will make HBcAg-specific T cell immune response weaken.

For the appearance of J group, the coexistence curve is promoted to move upper right, so that the C group with high affinity in the right side decreases the formation of pMHC-TCR. What kind of biological activity will the pMHC-TCR formed by J Group produce? When the number changes more than a degree in the mutual selection, due to the restriction by the constant of co-existence, there can determine the structure must change. However, when this new structure forms, it is unknown what the new function or biological activity it will bring with. The immune mutual selection between pMHC and TCR only shows the relationship that the two can not co-exist. They can not co-exist and lead the formation of pMHC-TCR, but it is unknown what kind of function this structure will bring about in theory.

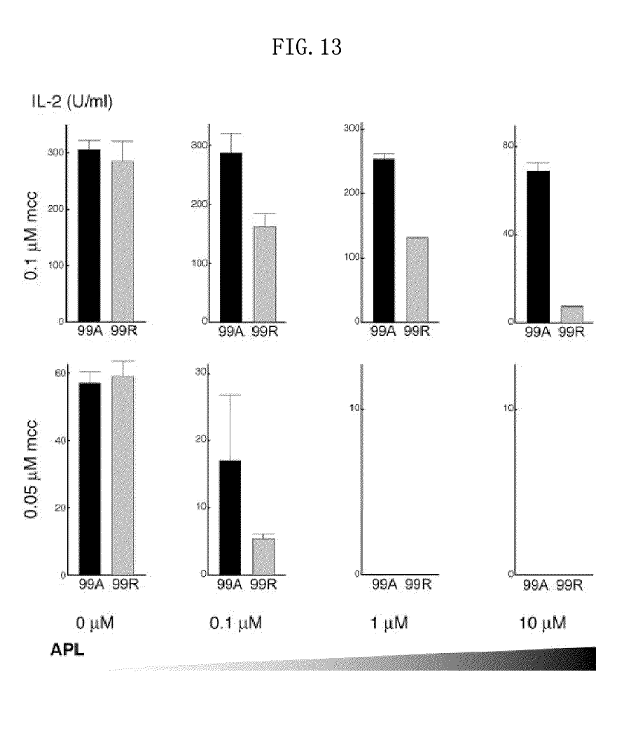

In the article written by Cenk Sumen, etc., the author provided the answer. According to the different affinity, the alterd peptide ligands (APLs, are variants of wild-type TCR-activating peptides that produce a range of effects from differential cytokine production to null peptides with no activity) of TCR can be divided into: null peptides, affinity not detected; antagonist peptides, low affinity; agonist peptides, a higher affinity. They used moth cytochrome c (MCC; 88-103) as agonist pMHC, and either of the single amino acid peptide mutants K99R (antagonist) or K99A (null) in their studies. They put 10 times of k99R and K99A to participate in the proliferation of MCC-mediated response, and MCC-mediated proliferation was markedly reduced (FIG. 12). The density of the central MHC in the immune synapse formation was decreased. Meanwhile, the APC with the constant density of MCC-MHC on cell surface, added k99R and K99A, can decrease the IL-2 concentration produced by T cells (FIG. 13). This effect starts to appear when their amount is 10 times of the MCC, 100 times more obvious, especially K99R. The article mentioned that antagonist peptides and mull peptides (pMHC) can participate in the immune synapse formed by agonist peptides, and reduce the number of MHC in the synapse, and play a role in blocking activation of signal transduction.

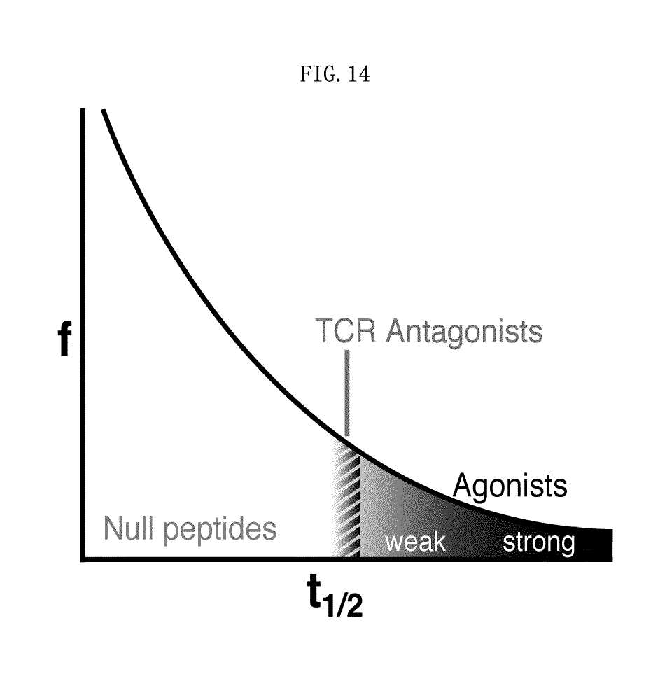

The photo (FIG. 14) in the discussion of the article, under the effect of a particular TCR, a group of pMHC with affinity from low to high, from left to right distributes “null peptides”, “TCR antagonists”, “weak agonists” and “strong agonists”.

The affinity referred in the patent between pMHC and TCR on the cells is on behalf of the affinity between cells. There are three affinity measurement methods between pMHC and TCR (David K. Cole, Nicholas J. Pumphrey, et al. Human TCR-Binding affinity is governed by MHC Class Restriction. J. Immunol. 2007; 178; 5727-5734.). The affinity represented by the half-life T1/2 of the interaction of cells through the pMHC-TCR has the most relevant of T cell activation. According to the affinity between the pMHC and the TCR from low to high, from left to right, the author arrangements in gradual change order as “null peptide”, “antagonist peptide”, “low agonist peptide” and “high agonist peptides”. Because of their experiments with the actual measurement through TCR and pMHC on the similar cellular system, their results should be restricted by the equilibrium constant of the mutual selection. They got a hyperbola, consisting with the theory, which shows the mutual selection theory in dealing with the interaction between pMHC and TCR is realistic. But they might consider many types of peptide-MHC. The frequency of peptides with high-affinity were less, while the frequency of peptides with low affinity were more, which is the reason that the curve in FIG. 14 is similar to the normal distribution of a group of peptides with a TCR in non-coexistence system, but this figure is only right part of the normal distribution curve. This is the contradiction of the theory and the practical measurement.

According to FIG. 11, the J group can form a large number of “null peptides”, “antagonist peptides” and “weak agonist peptides”. While these peptide-MHC complexes are distributed in the same APC, the “null peptides” and “antagonist peptides” will play a significant inhibition in the formation of the immune synapse induced by “strong agonist peptides”.

In summary, J Group peptides mainly reduce the number of TCR-pMHCs formed by the C group of peptides and inhibit the transmission of activation signals in the immune synapse formed by MHC-peptide of C group-TCRs.

In the above description, the high-affinity TCR is about the HBcAg. If the inhibition of J group can be enlarged to any pMHC-TCR with high affinity, can transcend any individual MHC or the MHC restriction of different species, the peptide or the peptides mixture like J group can reduce the formation of the specific high affinity MHC-pathogen peptide-TCR, which mediates T cell activation, proliferation and effect. The affinity of the peptides is between MHC-self-peptide-TCR and the specific high affinity MHC-pathogen peptide-TCR, which is known as T cell immune balance peptides.

4, T Cell Immune Balance Peptides

T cell immune balance peptides need not to consider the individual genetic background as the MHC restriction. According to the affinity to any one TCR, the pMHC group or the main part of the group formed by them is in the middle of the high-affinity pMHC (or group) and the individual's own pMHC group, which can negatively regulate the T cell immune response induced by the high-affinity pMHC (or group). The same peptide-MHC has different affinity with the different TCR in individuals, and its location in the q-axis is uncertainty. When the same peptide is in another individual, because the MHC and TCR are different, its affinity will be greater variation. However, the peptides mixture as a whole, its affinity in the q-axis position is determinable. In theory, there may be able to find a single peptide, and its position in the q-axis is relatively constant with a low affinity, which is consistent with the requirements of the balance peptides, but this method is not the pursuit of this patent. This patent is pursuit the certainty of the overall peptides, while a single peptide is uncertain.

(1) In Random Sequence

For the severe T cell specific immunity caused by viruses and bacteria mediated by MHC-pathogen peptide-TCR with high affinity, its pMHC group must be on the far right of the q-axis. The affinity of the pMHC group formed by the random peptides and MHC must be in the middle of the q-axis and in the left of the high affinity pMHC. In Cenk Sumen's article, most peptides they used are synthesized by chemical methods. They mentioned that the frequency of null peptides relative to any one of the TCR is always much more than the frequency of the activate peptides. Therefore, to any one of the pMHC-TCR with high affinity, the main (or mean) frequency distribution of the pMHC group is located in the left of q-axis, which is formed by the synthetic random peptides. In order to rule out the chance, the more types of the peptides, the better.

(2) Extracted from the Vertebrate Proteins

From the beginning of vertebrate animals, the special structure of pMHC-TCR is formed in immune system. Their structures may have differences, but they are similarities. Their proteins in the body are reserved by this selection system. The pMHC is formed form their proteins relative to the proteins of viruses and bacteria, which should has much lower affinity between the pMHC and the human body TCR in the whole. According to the occurrence of the living creature in the phylogenetic tree, the exogenous has a relationship with the degree of the differences among the genes of the species. If the antigen is from the farther phylogenetic species, its exogenous is more prominent and the immunogenicity is stronger. So the pathogen generally has strong immunogenicity in human bodies. The closer, the lower affinity, the immunogenicity is weaker; the farther, the higher affinity, the immunogenicity is stronger. Therefore, we can extract peptides from the vertebrate proteins, forming T cell immune balance peptides.

These are two sources of T cell balance immune peptides, and the sequence is diversity, and the length is the open. They usually need contain the peptides with the length covering class I and II molecules. Peptides Longer or shorter than them can be added, but the molecular weight should not exceed more than 10,000. The class I epitope may choose the peptides with 8˜10 amino acid residues, also can be the peptides only with eight or nine or 10 amino acid residues, also can choose a combination of any two lengths; II type epitope may choose the peptides with 13 to 25 amino acids residues, also can choose one of the length, two or more lengths of the combination. According to ease of the manufacture and pharmaceutical research, the peptides with 8 to 25 amino acids covering class I and class II epitope are preferred. The shorter peptides should be good to manufacture T cell immune balance peptides, such as I type epitope of the peptides with 8 to 10 amino acids, II-type epitope of the peptides with 13 to 14 amino acid residues. The molecular weight of the peptides can be estimated according to the number of amino acid residues and the type of amino acid using the existing formulas.

5, The Role of the T Cell Immune Balance of Peptides in T Cell Specific Immune Response

(1) The Binding Process Between T Cell Immune Balance Peptides and MHC Makes Effect on the Combination Between MHC and the Peptides from Specific Antigen

In the mutual selection between MHC and peptides, except the peptides with the molecule length of I and II epitopes, the longer peptides can be hydrolysis or enzymatic into suitable length in the lysosomal body. The peptides less than 8 amino acids may also work on MHC. At different affinity sites, they jointly promote the coexistence curve move up and right, resulting in reducing the combination of specific pathogen peptides and MHC, thereby reducing the formation of pathogenic peptide-MHC. From another point of view, it may contribute to the backlog of the pathogenic peptides, slow down the present of the pathogenic peptides, and extend the present time, so that the time for the pathogen peptides to induce specific immune response become longer.

The peptides extracted from pig's liver, named Cu Gan Xi Bao Sheng Zhang Su(PHGF) enhance expression of HLA-DR on the monocyte with different degrees for most people, and the health people are more significant (Qinghui Zhu, Yijun Zhang. pHGF on human monocyte—macrophage HLA—DR expression, Shanghai Journal of Immunology, 1996, No. 01), indicating that the peptides in PHGF bind to MHC in the human body and form a large number of pMHC expressed on the surface of the lymphocytes.

(2) The Effect of T Cell Immune Balance Peptide-Mhc Pairs in the T Cell Specific Immune Response

The peptides getting into the human body are likely to be captured by granulocyte macrophage, DC and B cells, and mainly be presented by class II molecules via the lysosome body way. They mainly impact in the activation phase. Some peptides may also be directly integrated with II molecules on the cell surface. Although the peptides can not rule out to be presented by class I molecules in cross-channel, which may affect the activation of CTL, but they are still in activation phase. Pinocytotic function of the parenchymal cells in human body is poor, and the peptides are unlikely to be presented in the cross-way. In addition, the infected cells are rich in pathogen proteins, and MHC may be mainly occupied by them.

In addition, the peptides may be able to directly bind to the cell surface MHC, as many studies use CTL epitope peptides directly stimulate the lymphocytes to produce specific immune response. However, the expression of class I molecules is highest in lymphocytes. A cell can contain 5×10⁵ molecules, about 1% of membrane proteins. M, DC, and neutrophils are also highly expressing HLA I molecules. On the contrary, lung, heart, liver cells, fibroblasts, muscle cells, nerve cells are expressing low levels of class I molecules. The class I molecules generally bind the peptides with 8 to 10 amino acids. T cell immune balance peptides binding to the empty MHC on the cell of the target organ are unlikely to mediate cytotoxicity.

Therefore, T cell immune balance peptides make a direct effect on the inhibition of the activation process in the activated phase of T cell specific immune response. By reducing the activation, so as to reducing the number of Th1 and CTL, thus makes the frequency of the specific TCR reduce. At the same time, the cytokines such as IFN-gamma are reduced. The present of pathogen peptide-MHC is reduced. So it reduces the number of the two parties in the mutual selection, which indirectly reduces the range, speed and strength of the CTL specific cytotoxicity in the effect phase.

T cell immune balance peptides are presented by the II molecules and form the low affinity pMHC, which competes with the pathogen pMHC to the pathogen-specific TCR on the same APC. It reduces the number of pathogenic pMHC binding to the specificity TCR, and reduces the formation of immune synapse guided by them. While the specific immune synapse has been formed, it inhibits the transmission of the activation signal, reducing the activation of T cells, the corresponding cytokine production and the proliferation of specific T cells. So they can reduce the intensity of the specific cellular immunity guided by Th1 cells and the specific humoral immunity guided by Th2 cells. The mutual selection mediated by pMHC-TCR is just the microscopic part of the mutual selection between the cells. The mutual selection between the cells makes changes in cell structure, so that the function changes.

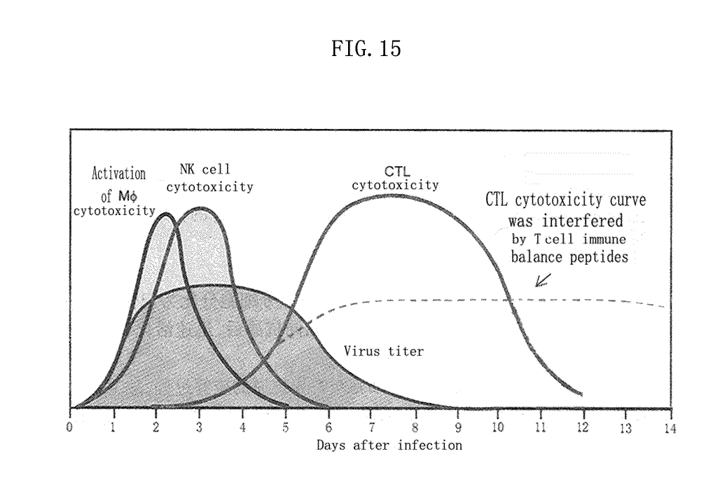

Summary, T cell immune balance peptides can interfere pathogenic peptides binding with MHC, and reduce the presentation in a time, extend the time for the presentation of the antigen peptides, and let T cell specific immune response be prolonged. The formation of pMHC formed by the T cell immune balance peptides is mainly composed of the class II molecules, and it reduces the binding between the formed pathogenic peptide-MHC and the specific TCR with high affinity. In the meantime, the pMHC formed by the T cell balance of peptides gets into the formation of immune synapse, which is mainly composed of the pathogen peptide-MHC and specific TCR. They suspend or reduce the activation signaling, decrease T cell activation, proliferation and cytokine production, and let T cell mediated pathogen specific immune response become weak. The time for the pathogen proteins to be presented is extended, and let specific immune reaction get longer. The curve of T cell-specific immune response changes from a peak shape into a platform, and the specific immune response becomes persistent and more moderate, as shown in FIG. 15.

The peptides mixture was composed by LLNQHACAV, env-236CTL epitope, pol-538CTL epitope, env313 CTL epitope, 88-96YVNTNMGLK, pol354-363 TPARVTGGVF, 18-27 FLPSDFFPSV, 141-151 STLPETTVVRR, padre AKFVAAWTLKAAA and pol501-515 LHLYSHPIILGFRKI, each peptide in the concentration of 20 μg/ml. They can inhibit the frequency of the IFN-gamma production cells, when the peripheral blood mononuclear cells were induced by HBcAg (20 μg/ml) in ELISPOT method (P<0.05). PHGF extracted from pigs' livers can play a more prominent role, showing a clear dose-effect relationship. This is one aspect that the T cell immune balance peptides can negatively regulate the specific immune reaction.

6, The Role of the T Cell Immune Balance Peptides in Normal Immune System

(1) The Promotion Function on the Phagocytic Cells in Innate Immunity.

The low molecular weight peptides extracted from the pig's liver, named Cu Gan Xi Bao Sheng Zhang Su(PHGF), were given intraperitoneally to mice at a dose of 1 mg, once daily for 5 consecutive days. Measured by chemiluminescence assays, the chemiluminescence rate of the mouse peritoneal cells in the experimental group was significantly higher than the control group after phagocytosis of bacteria, and the peak time was significantly shorter. The agent also significantly enhanced the mouse peritoneal macrophages to produce a factor with promoting procoagulant activity, thus the agent has a significant role on promoting macrophage function (Huaqing Chen, Yijun Zhang. 1996. The effect of pHGF on promoting the phagocytosis of human neutrophils. Shanghai Journal of Immunology. No. 01.). That enhancing the phagocytosis function of the phagocytic cells is an important cornerstone to use T cell immune balance peptides in infection diseases, which is beneficial to eliminate pathogens and prevent the occurrence of the concurrent infections.

(2) Induce a Weak Specific Immune Response, but Incompletely

T cell immune balance peptides mixed by a large number of peptides are obscured individual differences in MHC and TCR, so it needs not to consider the individual differences and the genetic background. A peptide in T cell immune balance peptides has a low repetition rate, thus makes the formation of the same pMHC with low repetition frequency. So the co-existence curve is at the upper right of the coordinates in the mutual selection between the same pMHC and all the TCR in individual, which is far away from the axis. Thus reduces their interaction region with the distribution of the TCR frequency, and reduces the occurrence of the possibility of activating reactions. And the high-affinity MHC-peptide structure induced by the small amount peptides from the balance peptides, which is likely to be interfered by the other peptides in the balance peptides, when the immune synapses are forming in the T cell activation process.

It is generally considered the molecular weight of ideal antigen should be more than 100 kDa. The molecular weight of the antigen is less than 5˜10 kDa with poor immunogenicity. In existing peptide vaccine studies, the immunogenicity of the epitopes is generally weak. When they are used alone, it is difficult to cause a strong immune response. They induce peripheral blood mononuclear cells to produce IFN-gamma, usually be detected by the high sensitive detection method of ELISPOT, which is usually not detected by commonly used ELISA method. As the average molecular weight of each amino acid of the proteins or peptides in nature is about between 100 to 140 Dalton, the peptide with molecular weight 10000 Dalton, which corresponding amino acid residues is about 75. So, T cell immune balance peptides can be consisted of peptides with randomly arranged amino acid sequence and the molecules weight being within 0-10000, or the number of amino acid residues being within 2 to 75.

When the naive T cells have gotten the antigen recognition signal (the first signal) through the three components of “MHC-antigen peptide-TCR”, they can be activated only when the co-stimulatory signal (the second signal) is appearance. The second signal has various sources, mainly from the binding between the B7 molecules (ligands) on APC surface and the CD28 molecule (receptor) on T cell surface. Without the second signal, the transcriptional activation of many genes (such as the gene encoding IL-2) can not occur, and thus the T cells have the antigen recognition signal can not enter the stage of clonal proliferation and differentiation, showing anergy state. Still APC is expression with little or no co-stimulatory molecules. The peptides extracted from pig's liver, they may be possible to activate the TCR related to the immunity with the pig species in human body, and perhaps they can form such pMHC-TCR structure in the body. However, there is lack of pathogen associated molecular pattern peptide (PAMP) which is used to activate the innate immune pattern recognition receptors and promote a variety of co-stimulatory molecules and cytokine expression. For the lack of appropriate co-stimulatory molecules and cytokine environment, the immune response induced by the peptides is very limited. In addition, even they can activate the naive T cells, but the formed effector T cells do not have specific target organs. They are likely to mediate very little and dispersed cytotoxicity, but the cell deaths caused by the cytotoxicity may be minimal compared with the normal metabolism of the cells in the body. Used for many years, there is no report that PHGF has been found as the immune activator leading to autoimmune diseases, indicating its average affinity is still lower than the antigen mediated autoimmune. 7, the manufacture and use of T cell immune balance peptides

(1) Random sequence. Various peptides can be chemical synthesized to form the T cell balance peptides. They can be use as the negative regular of T cell immunity during the acute viruses or bacteria infections, and also they may be used for the treatment of autoimmune diseases.

(2) Extracted from animals organs, such as liver, heart, kidney and spleen from cattle, pig, sheep, etc. In order to improving the types of the peptides, they can be extracted from only one organ, or a variety of organs, and also a variety of organs of different animals, thus reducing the use dose of a single peptide. The peptides extracted from the protein are good in a decomposition way of applied physics cutting approach, and try not to use the protease with the specific restriction sites, for the treatment of viruses and bacteria infection, such as severe influenza, SARS, hand-foot-and-mouth disease, viral pneumonia, bacterial infections, etc., and also autoimmune disease. Two drugs in the Chinese market can be developed into T cell immune balance peptides. One is the peptides extracted from animal liver, named Cu Gan Xi Bao Sheng Zhang Su and the other one is the peptides extracted from animal heart, named Xin Ji Tai Su. Particularly the peptides extracted from human organs, as opposed to the peptides extracted from pig and cattle, may be more generally low affinity to the TCR of the human body, which are very suitable for the treatment of human autoimmune diseases. As the ethical issues, they can not be mass production, but they can be used to produce the template for copy.

(3) T cell immune balance peptides can be made by selecting some or all of the peptides extracted from animals to synthesize in chemical synthesis way after the chemical analysis of their elements. The peptides can be production by using prokaryotic expression in genetic engineering methods, if the chain of which is too long to synthesis. Based on pharmacodynamic and (or) clinical study, the random peptides mixture can develop into a peptides mixture with certain number of peptides, amino acid sequence and contents, thus form a fixed formula of T cell immune balance peptides.

(4) The broad affinity of the peptides (biological products generally have a wide range of affinity, so they have a wide selection roles). In the mutual selection with the human body, the peptides are variety and the low repetition rate of a single peptide used can reduce the selection intensity to a particular same body, so may reduce the toxicity. As the T cell immune balance peptides at the left side of the q-axis, while the expression of pathogenic protein is usually achieved μg, mg or above in human body, so the dose of T cell immune balance peptides should be higher as 100 times, or even more than 1000 times, reaching the level of mg and g, for pushing up the two coexistence curves that have been pushed far to the upper and right by the pathogenic peptides. The dose of the peptides mixture may range from 0.1 mg/kg˜50 mg/kg. In such a large dose of the request, the diversity of the peptides' types is particularly important, which is closely related to security. So T cell immune balance peptides should be consisted of 10 or above 10 peptides with different randomly arranged amino acid sequence. The PHGF extracted from animal organs once can be used intravenously at a dose of 360 mg or more, and patients are able to tolerate, which shows these drugs have a very reliable security. The peptides mixture preparation can be in the form of freeze-dried powder or water solution.

The invention will now be further described with reference to the following examples.

### Example 1

### Preparation of the T Cell Immune Balance Peptides Extracted from Animal Organs and Tissues

(1) Select the healthy piglets or no breast-feeding newborn calf, take the fresh liver, heart, kidney and spleen, after washing with the water for injection, cut into pieces or shredding about 24° C.;

(2) add sterile water for injection by weight or volume ratio of 1:1. Repeatedly homogenate at 4° C. for 10 minutes. The homogenate liquid is frozen after placed in 60 to 70 degree below zero, then quickly be warmed up to 85° C., 10 minutes, achieve thermal denaturation of protein molecules, and remove the macromolecular protein;

(3) Heat denatured, 4° C., 3000 r/min, centrifugation for 20 minutes, discard the pellet, get the supernatant;

(4) Make steps to ultra-filtration and virus inactivation: the ultra-centrifugation supernatant to ultra-filtration filter for classification. The first step of the ultra-filtration collects the components with molecular weight below 30,000 Dalton. The second step of the ultra-filtration collects the peptides with molecular weight below 10,000 Dalton. Then according to need, further ultra-filtration is to get different molecular weight fractions. The molecular weight of the peptides mixture can be select from 800 to 2000 Dalton, from 800 to 3000 Dalton, from 1000 to 2000 Dalton, from 800 to 1000 Dalton, or from 800 to 10000 Dalton, and it could also be select two intervals from 800 to 1000 Dalton and from 1300 to 1800 Dalton. Target component is warmed at 60° C. for 10 hours, inactivated virus;

(5) Filter with 0.45 um, 0.22 um membrane. The filtrate adds excipients, be qualified, moderate-packed, and freeze-dried.

### Example 2

### Prepared the T Cell Immune Balance Peptides by Chemical Synthesis

Preparation of Peptides Using Fmoc solid phase peptide synthesis, an amino acid protected by the Fmoc group on the α-aminoion was linked to an insoluble carrier through a support arm, then the α-amino de-protect by using solution washing of amino acids—arm—resin. The second amino acid pre-activated and α-amino protected was linked up through the coupling reaction. When the condensation reaction was complete, it was washed with the washing solution, repeat de-protected, coupled, until getting a target peptide. Finally, peptide—Arm—resin cracked. Peptide was purified by HPLC method. The crude peptide product was purified by C18 high-pressure column. Collection with the HPLC effluent was needed. The sample peak combined and desalinated, freeze-dried, got the refined peptide.

Using the synthesis of HBcAg (18-27) as an example:

(1) Fmoc-Val-Wang Resin Swelling and Take Off the Fmoc Protection

Weigh Fmoc-Val-Wang resin 62.5 g (150 projects, 0.8 mmol/g), put it into a dedicated reactor. Open the total power of the CS936 productive peptide synthesizer, opened the station, call the pre-edited peptides linking procedures. Soak with 700 ml DMF, full swelling the resin, dried by nitrogen. Add 700 ml of DMF solution containing 20% PIP, and shook 30 minutes in 25° C. Nitrogen blown off PIP, washed three times with DMF, dried by nitrogen.

(2) Preparation of the Fmoc-Ser (tBu)-Val-Resin

Added Fmoc-Ser (tBu)-0H57.5 g, HOBt20.5 g, DIC 50.6 g, 300 ml DMF, shook the mixture for 1 hour in 25° C. Dried by nitrogen, washed 3 times with DMF, dried by nitrogen.

Added 400 ml of DMF solution containing 20% PIP, and shook 30 minutes in 25° C. Nitrogen blown off PIP, washed three times with DMF, dried by nitrogen.

(3) Preparation of the Fmoc-Pro-Ser (tBu)-Val-Resin

Added Fmoc-Pro-OH50.6 g, HOBt20.5 g, DIC 50.6 g, 300 ml DMF, shook the mixture for 1 hour in 25° C. Dried by nitrogen, washed 3 times with DMF, dried by nitrogen.

Added 400 ml of DMF solution containing 20% PIP, and shook 30 minutes in 25° C. Nitrogen blown off PIP, washed three times with DMF, dried by nitrogen.

(4) Preparation of the Fmoc-Phe-Pro-Ser (tBu)-Val-Resin

Added Fmoc-Phe-OH 58.1 g, HOBt20.5 g, DIC 50.6 g, 300 ml DMF, shook the mixture for 1 hour in 25° C. Dried by nitrogen, washed 3 times with DMF, dried by nitrogen.

Add 400 ml of DMF solution containing 20% PIP, and shook 30 minutes in 25° C. Nitrogen blown off PIP, washed three times with DMF, dried by nitrogen.

(5) Preparation of the Fmoc-Phe-Phe-Pro-Ser (tBu)-Val-Resin

Add Fmoc-Phe-OH 58.1 g, HOBt20.5 g, DIC 50.6 g, 300 ml DMF, shook the mixture for 1 hour in 25° C. Dry by nitrogen, washed 3 times by DMF, dried by nitrogen.

Add 400 ml of DMF solution containing 20% PIP, and shook 30 minutes in 25° C. Nitrogen blown off PIP, washed three times with DMF, dried by nitrogen.

(6) Preparation of the Fmoc-Asp(OtBu)-Phe-Phe-Pro-Ser (tBu)-Val-Resin

Added Fmoc-Asp(OtBu)-OH 61.7 g, HOBt20.5 g, DIC 50.6 g, 300 ml DMF, shook the mixture for 1 hour in 25° C. Dried by nitrogen, washed 3 times with DMF, dried by nitrogen.

Added 400 ml of DMF solution containing 20% PIP, and shook 30 minutes in 25° C. Nitrogen blown off PIP, washed three times with DMF, dried by nitrogen.

(7) Preparation of the Fmoc-Ser(tBu)-Asp(OtBu)-Phe-Phe-Pro-Ser (tBu)-Val-Resin

Added Fmoc-Ser(tBu)-OH 57.5 g, HOBt20.5 g, DIC 50.6 g, 300 ml DMF, shook the mixture for 1 hour in 25° C. Dry by nitrogen, wash by DMF 3 times, dry by nitrogen.

Added 400 ml of DMF solution containing 20% PIP, and shook 30 minutes in 25° C. Nitrogen blow off PIP, wash three times with DMF, dry by nitrogen.

(8) Preparation of the Fmoc-Pro-Ser(tBu)-Asp(OtBu)-Phe-Phe-Pro-Ser (tBu)-Val-Resin

Added Fmoc-Pro-OH 50.6 g, HOBt20.5 g, DIC 50.6 g, 300 ml DMF, shook the mixture for 1 hour in 25° C. Dried by nitrogen, washed by DMF 3 times, dried by nitrogen.

Added 400 ml of DMF solution containing 20% PIP, and shook 30 minutes in 25° C. Nitrogen blown off PIP, washed three times with DMF, dried by nitrogen.

(9) Preparation of the Fmoc-Leu-Pro-Ser(tBu)-Asp(OtBu)-Phe-Phe-Pro-Ser (tBu)-Val-Resin

Added Fmoc-Leu-OH 53.1 g, HOBt20.5 g, DIC 50.6 g, 300 ml DMF, shook the mixture for 1 hour in 25° C. Dried by nitrogen, washed by DMF 3 times, dried by nitrogen.

Added 400 ml of DMF solution containing 20% PIP, and shook 30 minutes in 25° C. Nitrogen blown off PIP, washed three times with DMF, dried by nitrogen.

(10) Preparation of the Fmoc-Phe-Leu-Pro-Ser(tBu)-Asp(OtBu)-Phe-Phe-Pro-Ser (tBu)-Val-Resin

Added Fmoc-Phe-OH 58.1 g, HOBt20.5 g, DIC 50.6 g, 300 ml DMF, shook the mixture for 1 hour in 25° C. Dried by nitrogen, washed by DMF 3 times, dried by nitrogen.

Added 400 ml of DMF solution containing 20% PIP, and shook 30 minutes in 25° C. Nitrogen blown off PIP, washed three times with DMF, dried by nitrogen.

Wash by ethanol 3 times. Drain, put into nitrogen gas dryer vacuum dried in nitrogen, get the protected the HBcAg (18-27) resin 103 g.

(11) preparation of the Phe-Leu-Pro-Ser-Asp-Phe-Phe-Pro-Ser-Val

Take the protected HbcAg (18-27) resin 103 g into the cut peptide bottle, cooling while stirring, and added cut peptide reagents (TFA/HBr/TIS/EDT=700 ml/28 ml/43 ml/21 ml), Stire 4 hours in 25° C. Filter, vacuum concentration, the filtrate added anhydrous ether precipitation, collection of precipitation, washed with ether, P2O5 drying, the HBcAg (18-27) crude peptides were about 45 g after cut.

Purification: The HbcAg (18-27) crude 45 g was dissolved in 20 L water, purification, filtrated. The filtrate was purified by C18 column, mobile phase: 0.1MNH4Ac: acetonitrile (75-25), the flow rate of 300 ml/min. Collecting with the HPLC effluent was needed, the sample peak combined and desalinated, was finished 12.5 g (MW: 1154), the total yield was about 22%.

Other peptides were synthesized by the above method. The various peptides were dissolved in sterile PBS in a certain percentage ratio, sterilized by filter, added excipient, freeze-dried and got the T cell immune balance peptides.

### Example 3

### Synthetic Peptides Mixture and CU GAN XI BAO SHENG ZHANG SU (PHGF) Inhibit IFN-Gamma Induced by HBcAg Detected in ELISPOT Way

Purpose

Combined with HBcAg stimulation of human peripheral blood mononuclear cells (PBMC) produced IFN-gamma, different concentrations of synthetic peptides mixture and PHGF impact on the IFN-gamma production, in order to reflect their specific immune regulation of T cells.

Reagents

RPMI1640 medium and fetal bovine serum, lymphocyte separation medium, anti-human CD3, HBcAg, synthesis of 10 peptides (previously mentioned), PHGF, ELISPOT kit, solid heparin anticoagulant, double-distilled water, sterile to ionized water.

Methods

6 health people were fasting before blood sampling at least 6 hours. In sterile conditions, collected peripheral blood 5 ml by using the blood vessels with high-pressure sterilization, added 100 units of heparin anticoagulation, and gently shake to reach an anticoagulant effect. Each blood sample diluted with 5 ml RPMI-1640 (including double antibiotic, room temperature) and mixed, placed in two vessels filled with 5 ml lymphocyte separation medium in 2 equal Parts, being careful to keep the interface clear. 2000 rpm centrifugation for 20 minutes, the interface cell layer after drawing to wash twice by RPMI-1640 (including double antibiotic, room temperature), suspended in 1 ml RPMI-1640 (including double antibiotic, 10% FCS, room temperature). After red blood cells dissolved by iced acetic acid and counted, the concentration of the cells was adjusted to 2×10⁶/ml. The operation process should strictly sterilize, and gentle movements to reduce cell damage to ensure cell viability. Centrifugation for 20 minutes at 2000 r/min, get the PBMC. The stimulus samples were prepared by two-step dilution method. In first step, the stimulus samples were diluted to 10 times of the required final concentration, and then diluted into 2 times. Adding 4×10⁵ cells/well and the corresponding stimulus into 96-hole microplate blocked by IFN-γ monoclonal antibody and the calf serum. Repeated the same stimulus, 37° C., 5% CO2, incubated for 15˜20 h. After washing, successively added biotinylated anti-IFNγ polyclonal antibody and enzyme-labeled goat anti-biotin antibody and the Activator I, II, the color dots were read by the count instrument.

Results

| | Formation of |
|---|---|
| | the number of |
| Stimulants | IFN-γ spots |
| 1. Negative control(RPMI 1640) | 1.0 ± 0.89 |
| 2. Anti-CD3(0.2 μg/ml) | >200 |
| 3. HBcAg(20 μg/ml) | 26.54 ± 17.93*& |
| 4. pHGF(0.075 mg/ml) | 1.0 ± 0.89 |
| 5. pHGF(0.3 mg/ml) | 0.67 ± 0.82 |
| 6. pHGF(1.2 mg/ml) | 0.67 ± 0.82 |
| 7. HBcAg(20 μg/ml) + 10 peptides | |
| (each 20 μg/ml) | 14.67 ± 13.56& |
| 8. HBcAg(20 μg/ml) + pHGF(0.075 mg/ml) | 20.67 ± 11.12 |
| 9. HBcAg(20 μg/ml) + pHGF(0.3 mg/ml) | 17.17 ± 12.77* |
| 10. HBcAg(20 μg) + pHGF(1.2 mg/ml) | 5.67 ± 7.76* |

The formation number of IFN-γ spot, 9 and 10 group were significantly different in group 3, P * <0.05. 8, 9, 10 group showed some dose-effect relationship. Group 7 and Group 3 was significantly different, P * <0.05.
The peptides mixture and PHGF worked as a negative regulator on HBcAg induced antigen-specific immune response.

### Example 4

### PHGF Inhibit IFN-Gamma Producing Cells Formation in PBMC by Flow Cytometry Analysis

Methods:

(1) Isolated PBMCs: blood samples were 10 ml, heparin 30 U/ml, mixed with RMPI 1640 basic medium 1:2, the blood got slowly along the wall superimposed on Ficoll lymphocyte separation medium (density: 1.077±0.002) with volume ratio 1:1, centrifuged to get the PBMCs. And then washed twice with RMPI 1640 basic medium, prepared cell suspension culture medium with 10% inactivated fetal calf serum RMPI 1640, counted cells. After using Trypan blue to calculate the cell viability, the cell viability required>95%, adjusted the cell concentration to 2×10⁶/ml.

(2) Activation: cells with the concentration of 2×10⁶/m1 were added to four 15 ml centrifuge tube cells, each tube 1 ml, as follows:

\textcircled{1} cells 10000

\textcircled{2} cells 10000+HBcAg (4 μg/ml)

\textcircled{3} cells 10000+HBcAg (4 μg/ml)+PHGF (0.3 mg/ml)

The culture was homogeneous mixture, 37° C., 5% CO2 incubated 24 h. Cells after treatment, the IFN-γ+T-cells and IFN-γ+NK cells were detected by flow cytometry analysis

Results:

Stimulated for 24 h, the percentage (%) of IFN-γ secretion cell in
human peripheral blood mononuclear cells (n = 6)

| | stimulus in vitro | | |
|---|---|---|---|
| | PRMI-1640 | | HBcAg + |
| Cell types | medium | HBcAg | PHGF |
| CD3⁺/IFN-γ⁺ | 0.035 ± 0.029 | 0.091 ± 0.039* | 0.044 ± 0.046* |
| CD56⁺/IFN-γ⁺ | 0.034 ± 0.048 | 0.064 ± 0.037 | 0.054 ± 0.033 |

T cells stimulated with HBcAg + PHGF compared stimulated with HBcAg alone, IFN-γ + positive cells were significantly decreased, P * <0.05;
NK cells stimulated with HBcAg + PHGF compared stimulated with HBcAg alone, IFN-γ + positive cells were decreased, but not significantly.

### Example 5

According to the method of example 1, extracted the peptides with molecular weight range of 800˜2000 from pig or human liver, separated the peptides with the number of amino acids is 8, 9, 10, 13 and 14. Select 2 to 20 peptides of each length for the preparation of T cell immune balance peptides. After the analysis of their Amino acid sequence, the peptides were synthesized according to the method of Example 2. According to the weight ratio of 1:1, the peptides mixture was lyophilized and then T cell immune balance peptides were prepared. Preference for 10 peptides of each length for preparation, the T cell immune balance peptides were composed of 50 different peptides and the weight ratio of each peptide is 1:1. The peptides mixture used to prepare the drug for the treatment of viruses, bacteria and other microbial infections and autoimmune disease.

I claim:

1. T cell immune balance peptides are consisted of 10 or above 10 peptides with different randomly arranged amino acid sequence and the molecules weight being within 0˜10000, or the number of amino acid residues being within 2 to 75.

2. According to claim 1, the peptides mixture has the following characteristics: without the need to consider the patients' genetic background, it can interfere with the T cell specific immune response induced by microorganism (virus, bacteria, etc.), protein and other macromolecular antigens, especially when the response is very strong.

3. According to claim 2, the interference of the peptides mixture can reduce the specific immune cell activation, proliferation and effect, reduce the production and secretion of various cytokine, and make the radical T cell specific immune response more stable and last longer.

4. According to claim 2, the interference of the peptides mixture against T cell-mediated specific immune response mainly works through interfering MHC-pathogenic peptide-TCR formation, including interfering pathogenic peptides binding to MHC and pMHC binding to specific TCR; Alternatively, it acts by suppressing the immune synapse formation during pathogen-specific immune response, decreasing the number and density of MHC-pathogenic peptide-TCR in immune synapse, and interfering with the highly-activated signal transduction in the immune synapse.

5. According to claim 1, the molecular weight of the peptides mixture includes any single or combined intervals in the region 0-10000 and it only excludes the interval 0-700 or any sub-intervals within it.

6. According to claim 1, the number of the amino acid residues of the peptides is 8, 9, 10, 11, 12, 13, 14, 15, 16, 17, 18, 19, 20, 21, 22, 23, 24, 25.

7. According to claim 1, the peptides mixture can be composed by the peptides with 8, 9, 10, 13 and 14 amino acid residues.

8. According to claim 7, each of the five peptides with different number of the amino acid residues can select 2 to 20 peptides with different sequences, and priority to choice 10 peptides; the weight ratio of each peptide is 1:1.

9. According to claim 5, the molecular weight of the peptides mixture can range from 0 to 10000 Dalton, from 800 to 2000 Dalton, from 800 to 3000 Dalton, from 1000 to 2000 Dalton, from 800 to 1000 Dalton, or from 800 to 10000 Dalton, and it could also be two intervals from 800 to 1000 Dalton and from 1300 to 1800 Dalton.

10. According to claim 1, the peptides mixture can be synthesized by chemical methods and (or) the expression of genetically engineered bacteria.

11. According to claim 1, the peptides mixture can be extracted from vertebrate animals with rich protein.

12. According to claim 1, based on pharmacodynamic and (or) clinical study, the random peptides mixture can develop into a peptides mixture with certain number of peptides, amino acid sequence and contents, thus form a fixed formula of T cell immune balance peptides.

13. According to claim 1, the peptides mixture preparation can be in the form of freeze-dried powder or water solution.

14. According to claim 1, the dose of the peptides mixture ranges from 0.1 mg/kg˜50 mg/kg.

15. According to claim 1, the peptides mixture is given via intravenous.

16. According to claim 1, the peptides mixture is used to prepare the drugs for the treatment of diseases with excessive T cell-specific immune response which leads to the pathological processes, such as severe influenza, SARS, hand-foot-and-mouth disease, viral pneumonia, bacterial infections, severe autoimmune disease and etc.

17. According to claim 11, two drugs in the Chinese market can be developed into T cell immune balance peptides; one is the peptides extracted from animal liver, named Cu Gan Xi Bao Sheng Zhang Su and the other one is the peptides extracted from animal heart, named Xin Ji Tai Su.

18. According to claim 11, the T cell immune balance peptides can be extracted from human internal organs.

19. According to claim 18, the peptides mixture can be prepared by chemical synthesis or the expression of genetic engineering method, based on the chemical composition analysis using part or all of the composition extracted from human body proteins.

20. According to claim 19, the peptides mixture is used to prepare the drugs for the treatment of human autoimmune diseases.


SEQUENCE LISTING (7 sequences)

SEQ ID NO: 1 - protein, 9 aa, Homo Sapiens
  CHAIN (218)...(226): AFP
LLNQHACAV

SEQ ID NO: 2 - protein, 9 aa, hepatitis B virus
  CHAIN (88)...(96): HBcAg
YVNVNMGLK

SEQ ID NO: 3 - protein, 10 aa, hepatitis B virus
  CHAIN (354)...(363): HBV polymerase protein
TPARVTGGVF

SEQ ID NO: 4 - protein, 10 aa, hepatitis B virus
  CHAIN (18)...(27): HBcAg
FLPSDFFPSV

SEQ ID NO: 5 - protein, 11 aa, hepatitis B virus
  CHAIN (141)...(151): HBcAg
STLPETTVVRR

SEQ ID NO: 6 - protein, 13 aa, artificial sequence
  PADRE
AKFVAAWTLKAAA

SEQ ID NO: 7 - protein, 15 aa, hepatitis B virus
  CHAIN (501)...(515): HBV polymerase protein
LHLYSHPIILGFRKI